(19) United States
(12) Patent Application Publication
(10) Pub. No.: US 2009/0061491 A1
(43) Pub. Date: Mar. 5, 2009

(54) Novel fermentation method

(76) Inventors: Ulrike Becker, Weil am Rhein (DE); Karlheinz Bretz, Rodersheim-Gronau (DE); Udo Koller, Lorrach (DE)

Correspondence Address: NIXON & VANDERHYE, PC, 901 NORTH GLEBE ROAD, 11TH FLOOR, ARLINGTON, VA, 22203, US

(21) Appl. No.: 11/921,505

(22) PCT Filed: May 31, 2006
(86) PCT No.: PCT/EP2006/005166

§ 371 Date: Jun. 13, 2008

(30) Foreign Application Priority Data
Jun. 6, 2005 (EP) 05012131.8

(51) Int. Cl. C12P 1/04; C12P 17/12

(52) U.S. Cl. 435/122; 435/170

(57) ABSTRACT

This invention relates to a process for recycling biomass in fermentation processes, whereby the biomass that accumulates in the production of fermentation products is prepared by means of a special process and is recycled into the system. The prepared biomass can be used again in the fermentative production of vitamins, in particular vitamin B2, as a medium component in fermentation.

This invention relates to a process for recycling biomass in fermentation processes, whereby the biomass that accumulates in the production of fermentation products is prepared by means of a special process and is recycled into the system. In the fermentative production of vitamins, in particular vitamin B2, the prepared biomass can be used again as a medium component in the fermentation.

In the meantime, many commercially valuable products, including vitamins, are produced by fermentation. One example of this is vitamin B2 (riboflavin), which is an essential vitamin for all bacteria, animals and plants. In contrast to plants and bacteria that can synthesize riboflavin themselves, it must be fed by food in the case of higher animals, such as, e.g., vertebrates.

After a fermentation process is completed, normally first the fermentation product is isolated from other components, such as, e.g., the biomass, and optionally further purified, which can result in losses in yield of the fermentation product. The accumulating biomass, although very high in energy, in most cases goes unused as a waste product and must be removed (stored, composted, burned) at the expense of considerable funds and resources.

Another disadvantage of the known fermentation processes is the high cost of the fermentation itself, brought about by feeding raw materials to the culture medium. Thus, for example, yeast extract is used for better growth of the microorganisms.

The object of this invention was to develop an improved fermentative process that on the one hand results in saving costs and resources, but on the other hand also guarantees optimal yields and purity of the fermentation product.

It was now possible to show, surprisingly enough, that by using prepared biomass (“biomass recycling”) as a medium component in the fermentation, the addition of yeast extract can be eliminated. In addition, it was possible to increase the purity and yield of the fermentation product by the selected conditions during the preparation process.

This invention is directed in particular toward a process for recycling biomass into fermentative processes.

The terms “recycling of biomass” and “recycled biomass” are used here in terms of prepared and recycled biomass or biomass that is used again. The biomass that accumulates in a fermentation process is prepared under suitable conditions so that it can be recycled (fed back) into the system, i.e., the fermentation process, again. In this case, the preparation includes isolation and decomposition as well as optionally additional purification and concentration steps. The recycled biomass can replace other medium components that normally are fed to the culture medium for better growth of microorganisms. An example of such a component that can be replaced by the recycled biomass is yeast extract.

In addition, this invention relates to a process for fermentative production of fermentation products, such as, e.g., vitamins, by means of/using recycled biomass.

This process can be used for all vitamins that are produced by fermentation. Preferred vitamins are the vitamins of the B complex or biotin. Preferred vitamins of the B complex are riboflavin, pantothenic acid, thiamine, folic acid and pyridoxine, including their salt forms and derivatives. Especially preferred is riboflavin, including its derivatives, such as, e.g., riboflavin-phosphates. Products in terms of this invention, i.e., vitamins produced via fermentation, are also referred to as “fermentation products.”

Fermentation processes for the production of vitamins according to the invention can be performed in batch, fed-batch, continuous or semi-continuous processes. The process can include cells from microorganisms, in particular recombinant microorganisms, being cultivated under suitable growth conditions, e.g., by inoculating a microorganism in a suitable medium so that it contains all necessary substrates for growth of the microorganism and for the production of the fermentation product. The inoculated medium is subjected to specific physico-chemical parameters, such as, e.g., temperature, pH and aeration, which allow optimal growth of biomass and accumulation of the fermentation product. Depending on the microorganism that is used and the product to be produced, these parameters can vary greatly. Thus, e.g., fermentation can occur under aerobic or anaerobic conditions. Processes for empirical determination of these parameters are known to one skilled in the art.

After the fermentation is completed, the resulting cell extract, containing, i.a., biomass and the desired (unpurified) fermentation product, can be isolated from the fermentation broth and decomposed again, e.g., can be further processed in a thus mentioned “downstream processing.” Methods for isolation and purification are known to one skilled in the art. Thus, the fermentation product can be isolated from the medium and other components, such as, e.g., the microorganism. In terms of this invention, “isolated” is defined as, e.g., the product that is purified or at least partially purified by means of, for example, filtration, centrifuging and/or extraction. The subsequent purification of the product can be carried out, e.g., via recrystallization from aqueous to organic solvents or by using additional methods that are known to one skilled in the art, thus, e.g., ion exchange, size exclusion or chromatography via hydrophobic interaction.

In the case of riboflavin that is produced by fermentation, whereby said riboflavin is precipitated in the form of crystals in culture medium, the isolation from the fermentation broth can be carried out by means of centrifuging. The riboflavin crystals are then purified in a known way; see also in this connection EP 730034 A1 or Bretzel et al., Journal of Industrial Microbiology & Biotechnology (1999) 22, 19-26.

The terms “purification,” “decomposition,” and “preparation” in connection with cell extracts including fermentation products and biomass are used synonymously in this application.

Known methods for preparing cell extracts resulting from fermentation are, e.g., physical, chemical or biological decomposition methods. A chemical decomposition includes, e.g., treatment with strong acids or bases, solvents or detergents. In the case of a physical decomposition, a distinction is made between mechanical methods, e.g., ultrasound or pressure (high-pressure homogenization), and non-mechanical methods, such as, e.g., osmotic shock, plasmolysis, freezing/thawing or thermolysis. Biological methods comprise the treatment with enzymes, antibiotics, phages or autolysis.

After the fermentation is completed, the preparation of the cell extract according to the invention can be performed by means of physical, chemical or biological decomposition. Autolysis is preferred. The term “cell extract” in terms of this invention relates to an extract of cells resulting from a fermentation process, which is present in unpurified form and can contain, i.a., the biomass and the fermentation product.

Preparation of the cell extract, e.g., by means of autolysis, can be carried out immediately in fermentation, i.e., decomposition is carried out immediately from the fermentation broth. In this case, the cell extract that is to be prepared contains, i.a., biomass and the fermentation product. It is also possible to isolate the biomass from the fermentation broth and/or to concentrate it after completing the fermentation, first by means of methods known to one skilled in the art, before the preparation of the biomass, e.g., by means of autolysis, is carried out.

In a preferred embodiment of the invention, the preparation of the cell extract is carried out immediately in connection to the fermentation, i.e., without previous separation of the fermentation product, such as, e.g., riboflavin. In this case, the preparation, preferably autolysis, is induced immediately in connection to fermentation. The induction can be carried out by, for example, changing the temperature.

In one embodiment of the invention, the preparation of the cell extract is performed by means of autolysis. The period of autolysis can be between about 1 hour and several days, in particular about 8 hours, 16 hours, or 24 hours, based on the microorganism and the conditions. By measuring the cell dry mass (ZTM), the degree of lysis can be determined, whereby the autolysis can be completed if the values for the ZTM remain constant. The lower the measured ZTM, the higher the degree of lysis. Methods for measuring the ZTM are known to one skilled in the art. The autolysis is preferably performed for about 24 hours.

A suitable pH in the case of autolysis can be about 6.0 to about 9.0, preferably at about 6.0 to about 8.5, in particular at about 6.5 to about 8.0. A pH from about 6.5 to about 7.5 is especially preferred. Optimal results are achieved at pH values of 6.5 and 7.5.

A suitable temperature in the case of autolysis can vary within the range of about 30° C. to about 60° C., preferably from about 30° C. to about 50° C., especially preferably from about 35° C. to about 45° C. Optimal results are achieved at temperatures of about 40° C. to about 45° C.

In one embodiment of the invention, the preparation of the cell extract is performed by means of autolysis for about 24 hours at a pH of about 6.5 to about 8.0 and at a temperature of about 35° C. to 45° C.

In another embodiment of the invention, the preparation is performed by means of autolysis for about 24 hours at a pH of about 6.5 to about 7.5 and a temperature of about 40° C. and about 45° C.

To optimize the preparation of the cell extract, the autolysis can be performed in the presence of additional enzymes, such as, e.g., hydrolases or proteases (endo- and exoproteases). The amount of enzyme can be, for example, 1.5% of the ZTM; the addition can be carried out, for example, after about 1 to 5 hours starting from the beginning of the autolysis. In otherwise uniform conditions, the ZTM can be dropped by up to 50% by adding these enzymes. In addition, this results in elevated degrees of lysis up to 50% to a shortening of the lysis times and to an increase in the product quality, i.e., a higher purity of the fermentation product (if the autolysis is performed immediately in fermentation as described above). Non-limiting examples of such proteases are alcalase (Calbiochem), Amano N, Umimazyme, Amano P, Pepdidase R (all Amano Enzyme Europe Ltd) or Promod (Biocatalysts Ltd.).

In one embodiment of the invention, the preparation is performed by means of autolysis for about 24 hours at a pH of about 6.5 to about 7.5 and a temperature of about 40° C. and about 45° C. with use of added proteases. A mixture of endo- and exoproteases is preferred.

The recycled biomass (prepared biomass extract) can be used in the fermentative production of vitamins.

This invention thus relates to a process for fermentative production of a vitamin, in particular riboflavin, containing (a) the fermentation of a microorganism under conditions and in a suitable medium that allow the production of the fermentation product and (b) the preparation of the biomass, in particular by autolysis.

The biomass can be isolated/separated from the cell extract, produced as described above, from the residual biomass and the fermentation product, and then further purified and/or concentrated. The purification of the lysate can be carried out by, e.g., crossflow filtration, and the concentration can be carried out by, e.g., a falling film evaporator. The thus produced, prepared biomass extract can be used/reused in fermentative processes as a medium component.

In terms of this invention, a (biomass) extract that is prepared by means of autolysis is also referred to as “lysate.” The prepared biomass extract or the lysate can be used in the fermentation process as a substitute for yeast extract, which normally is added to the culture medium for improved growth during fermentation. This means a recycling of the biomass after corresponding preparation as described above. Starting from the carbon content of yeast extract, which is known to one skilled in the art, the carbon content of the prepared biomass extract is determined by means of, e.g., a CHN-elementary analyzer. The yeast extract is thus replaced one for one (based on the carbon content) with the prepared biomass extract in the culture medium (fermentation medium).

This invention thus relates to a process for fermentative production of vitamins, in particular riboflavin, containing the following steps:
(a) Fermentation of a microorganism for the production of a vitamin, under conditions and in a suitable medium that allow the production of the vitamin,
(b) Preparation of the cell extract, in particular by autolysis, whereby preparation immediately follows fermentation,
(c) Isolation of the prepared biomass from the fermentation broth that contains vitamins and residual biomass,
(d) Purification and concentration of the prepared biomass, and
(e) Recycling of the (prepared) biomass extract into the fermentation process with repetition of steps (a) to (d), whereby the fermentation is performed without the addition of yeast extract in the culture medium.

Preferred embodiments that relate to steps (a) to (e) are described as above.

Suitable microorganisms for embodying this invention can be all microorganisms that are suitable for the fermentative production of the above-mentioned products, thus, e.g., for riboflavin. The microorganisms could be selected from the group that consists of Escherichia, Bacillus, Cyanobacter, Streptomyces and coryne bacteria. Recombinant and non-recombinant microorganisms can be used.

One example of a suitable microorganism for the production of riboflavin is Bacillus. A non-sporulating microorganism of the genus Bacillus, in particular B. subtilis, especially preferably B. subtilis RB50, is preferred. Most preferred is B. Subtilis RB50::(pRF69)ₙ::(pRF93)ₘ (Perkins et al., J. Ind. Microbiol Biotechnol 22: 8-18, 1999).

In the Agricultural Research Culture Collection (NRRL), Peoria, Ill., USA, on May 23, 1989, B. Subtilis RB 50 was deposited under No. NRRL B-18502 according to the Budapest Treaty. Plasmid pRF69 was deposited under No. ATCC 68338 on Jun. 6, 1990 in the American Type Culture Collection (ATCC), Rockville, Md., USA, according to the Budapest Treaty. Plasmid pRF93 is described in EP 0 821 063.

In one embodiment, this invention relates to a process for the production of riboflavin that contains:
(a) Fermentation of a microorganism of the genus B. subtilis, under conditions and in a suitable medium that allow the production of riboflavin,
(b) Autolysis at a pH of about 6.5 to about 7.5 and a temperature of about 45° C. for about 24 hours,
(c) Isolation of the prepared biomass from the fermentation broth that contains riboflavin and residual biomass,
(d) Purification and concentration of the prepared biomass, and
(e) Recycling of the B. subtilis lysate into the fermentation process with repetition of steps (a) to (d), whereby the fermentation is performed without adding yeast extract in the culture medium.

In another aspect of the invention, a process for the production of riboflavin is claimed, whereby by preparing the fermentation broth by means of autolysis, as described above, the yield and purity of the riboflavin that is obtained can be increased.

In particular, the invention relates to a process for the production of riboflavin, containing:
(a) Fermentation of a microorganism of the genus B. subtilis, under conditions and in a suitable medium that allow the production of riboflavin,
(b) Autolysis of the fermentation broth resulting from step (a) and containing riboflavin that is produced by fermentation,
(c) Isolation of the riboflavin and optionally
(d) Purification of the riboflavin.

Methods for isolating and purifying riboflavin are described above and are known to one skilled in the art. The autolysis is carried out under conditions as described above. Increases in yield of up to 2%, 4%, 6%, 10%, 20% or 30% can be achieved by this process, whereby the riboflavin concentrations are measured before and after step (b). In this case, a process in which the autolysis immediately follows the fermentation is preferred.

In a preferred embodiment, a process for the production of riboflavin is claimed, including:
(a) Fermentation of a microorganism of the genus B. subtilis, under conditions and in a suitable medium that allow the production of riboflavin,
(b) Autolysis of the fermentation broth that results from step (a) at a pH of about 6.5 to about 7.5 and a temperature of about 45° C. for about 24 hours,
(c) Separation of the prepared biomass and the riboflavin from the other components,
(d) Purification of riboflavin as well as purification and concentration of the prepared biomass, and
(e) Recycling of the B. subtilis lysate into the fermentation process with repetition of steps (a) to (d), whereby the fermentation is performed without adding yeast extract in the culture medium.

Optionally, the autolysis can be supported by using various enzymes, as described in more detail above.

Another aspect of the invention relates to the riboflavin-synthase, which catalyzes the last step of the biosynthetic production in B. subtilis, i.e., the conversion of 6,7-dimethyl-8-ribityllumazine (DMRL) to riboflavin. The above-described process results in an increase in the riboflavin-synthase activity. The activity can be measured by, for example, the conversion rate of DMRL into riboflavin.

In addition, the above-produced process for the production of riboflavin results in increasing the purity of the fermentation product, which can be measured by, e.g., a reduced DNA content in the riboflavin crystals. This is important in particular in the production of riboflavin by means of recombinant microorganisms. Methods for measuring the DNA content, such as, e.g., PCR, are known to one skilled in the art.

### EXAMPLES

### Example 1

### Cultivation Techniques and Analysis Process

For riboflavin production, the Bacillus subtilis strain RB50::(pRF69)ₙ::(pRF93)ₘ was cultivated for 48 hours in a glucose-limited medium by means of Fed-Batch fermentation (Perkins et al., J. Ind. Microbiol Biotechnol 22: 8-18, 1999).

The growth of the biomass was determined based on the dry weight of biomass (BTM). The dry weight of biomass was determined by the fermentation broth being cooled to room temperature. Then, 10 ml was added to a preweighed reagent glass and centrifuged off at 11,000 g. The supernatant was discarded, and the pellet was dried at 95° C. for at least 24 hours until a constant weight was reached. The BTM was produced from the specific GTM (total dry mass) minus the content of riboflavin (determination of the riboflavin content by means of HPLC).

### Example 2

### Production of an Extract by Means of Pressure (Physical Decomposition)

After completion of a riboflavin fermentation (see Example 1), the fermentation solution that was obtained was centrifuged off at 4250 g, and the biomass was isolated. The biomass that was obtained was washed three times with 50 mmol of sodium-potassium-phosphate buffer (pH 7.0), and the protein content and the BTM were examined. The biomass was decomposed in 3 passes at 1900 bar in a high-pressure homogenizer (Microfluidizer M110-EH, Microfluidics, Newton, Mass., USA) (2-nozzle system 1^{st} nozzle: 200 μm; 2^{nd} nozzle: 100 μm).

### Example 3

### Production of an Extract by Means of Autolysis (Biological Decomposition)

To determine the optimal temperature of autolysis, the fermentation solution obtained from Example 1 was incubated for 4 hours with a riboflavin content >20 g/l while being stirred and without introducing air at a constant pH of 7.0 and the temperature indicated in Table 1. Then, the degree of lysis was determined based on the BTM (see Table 1). An optimal autolysis was achieved at a temperature of 40-45° C.

TABLE 1
Autolysis Based on Temperature

| | Temperature [° C.] | | | | | | |
|---|---|---|---|---|---|---|---|
| | 30 | 35 | 40 | 45 | 50 | 55 | 60 |
| BTM [%] | 56.9 | 53.2 | 43.5 | 51.2 | 68.8 | 74.4 | 86.5 |

To determine the optimal pH of the autolysis, the fermentation solution obtained from Example 1 was incubated for 24 hours with a riboflavin content >20 g/l while being stirred and without introducing air at a constant temperature of 40° C. and the pH values indicated in Table 1. Then, the degree of lysis was determined based on the BTM (see Table 2). An optimal autolysis was achieved at a pH of 6.5- 7.5.

TABLE 2
Autolysis Based on the pH

| | pH | | | | | | | | |
|---|---|---|---|---|---|---|---|---|---|
| | 5.0 | 5.5 | 6.0 | 6.5 | 7.0 | 7.5 | 8.0 | 8.5 | 9.0 |
| BTM | 100 | 91.9 | 62.7 | 44.5 | 45.9 | 43.5 | 46.1 | 69.9 | 69.1 |
| [%] | | | | | | | | | |

A pH setting of 6.5 to 7.5 at a temperature of 40-45° C. is produced for an optimal autolysis from these results. To minimize the risk of contamination without using antiseptics, the temperature was also set at 45° C.

In another series of tests, the autolysis was tested in the presence of various proteases and their influence on the degree of lysis was examined.

The following enzymes were used: Alcalase (Calbiochem, San Diego, Calif., USA), Amano N, Amano P, Umimazyme, Peptidase R (all from Amano Enzyme Europe Ltd., Chipping Norton, U.K.) as well as Promod 194P (Biocatalysts Ltd., Treforest, U.K.).

The autolysis was performed as described above at a pH of 6.5 or 7.5 (45° C., 24 hours), with or without the above-described enzymes according to Table 3. Then, the degree of lysis was determined based on the BTM. An increase in the degree of lysis by a maximum of 20% was achieved by adding endoproteases during the lysis process (see Table 3).

### Example 4

### Production of Riboflavin by Means of Recycled Biomass Extract

Suspended matter was removed from the recovered extract from Example 3 by means of crossflow filtration (cutoff 10 kDa) and used in fermentation as in Example 1, whereby the yeast extract was replaced one for one based on the carbon balance. In this connection, the following results were achieved (see Table 3).

TABLE 3
Riboflavin Production by Means of Recycling of the Biomass

| | Riboflavin Concentration | |
|---|---|---|
| | [%] | Yield [%] |
| Standard (Yeast Extract) | 100 | 100 |
| Bacillus Lysate (from | 90.7 | 93.6 |
| Washed Biomass) | | |
| Bacillus Lysate (from | 78.7 | 77.7 |
| Unwashed Biomass) | | |

1. Process for recycling biomass in fermentative processes containing the preparation of a cell extract, resulting from fermentation, for recycling the purified biomass into the fermentation process.

2. Process for fermentative production of vitamins with use of recycled biomass produced according to claim 1.

3. Process according to claim 2, whereby the vitamins are selected from the group that consists of riboflavin, pantothenic acid, biotin, thiamine, pyridoxine and folic acid, in particular riboflavin.

4. Process according to claim 1, whereby the preparation of the cell extract is carried out by physical, chemical or mechanical lysis, in particular by autolysis.

5. Process according to claim 4, whereby the autolysis is performed for about 24 hours at a pH of about 6.0 to about 9.0, in particular of about 6.0 to about 8.5, and a temperature of about 30° C. to about 60° C., in particular of about 30° C. to 50° C.

6. Process according to claim 5, whereby the autolysis is performed for about 24 hours at a pH of about 6.5 to about 7.5 and a temperature of about 40° C. to about 45° C.

7. Process according to claim 1, whereby the preparation of the biomass is performed by means of autolysis in the presence of added proteases.

8. Process according to claim 1, whereby preparation of the biomass immediately follows fermentation without an intervening purification step.

9. Process according to claim 1, containing the following steps:
(a) fermentation of a microorganism for the production of a vitamin, in particular riboflavin, under conditions and in a suitable medium that allow the production of the vitamin,
(b) preparation of the biomass, in particular by autolysis, whereby preparation immediately follows fermentation,
(c) isolation of the prepared biomass from the fermentation broth that contains vitamins and residual biomass,
(d) purification and concentration of the prepared biomass, and
(e) recycling of the (prepared) biomass extract into the fermentation process with repetition of steps (a) to (d), whereby the fermentation is performed without the addition of yeast extract in the culture medium.

10. Process according to claim 9 for the production of riboflavin, whereby the preparation in step (b) is carried out for 24 hours by means of autolysis at a pH of about 6.5 to about 7.5 and a temperature of about 45° C.

11. Process according to claim 1, whereby the microorganisms that are used in the fermentation are selected from the group that consists of Escherichia, Bacillus, Cyanobacter, Streptomyces and coryne bacteria, in particular Bacillus.

12. Process according to claim 11, whereby the microorganism that is used in fermentation is Bacillus subtilis, in particular a non-sporulating B. subtilis.

13. Process according to claim 12, whereby the microorganism that is used in fermentation is B. subtilis RB50, in particular RB50::(pRF69)ₙ::(pRF93)ₘ.

14. Process according to claim 2 that contains:
(a) fermentation of a microorganism of the genus B. subtilis, under conditions and in a suitable medium that allow the production of riboflavin,
(b) autolysis of the fermentation broth, resulting from step (a), at a pH of about 6.5 to about 7.5 and a temperature of about 45° C. for about 24 hours,
(c) separation of the prepared biomass and the riboflavin from the usual components,
(d) purification of riboflavin as well as purification and concentration of the prepared biomass, and
(e) recycling of the B. subtilis lysate into the fermentation process with repetition of steps (a) to (d), whereby the fermentation is performed without adding yeast extract in the culture medium.
